(19) United States
(12) Patent Application Publication
(10) Pub. No.: US 2015/0313450 A1
(43) Pub. Date: Nov. 5, 2015

(54) Stopper for the Cables of a Bending Section of An Endoscope

(71) Applicant: EndoChoice, Inc., Alpharetta, GA (US)

(72) Inventors: Stephan Wieth, Klein Nordende (DE); Alexander Lang, Wedel (DE); Golan Salman, Atlit (IL); Amram Aizenfeld, Ramot Menashe (IL)

(21) Appl. No.: 14/702,676

(22) Filed: May 2, 2015

Related U.S. Application Data
(60) Provisional application No. 61/987,920, filed on May 2, 2014.

(51) Int. Cl. A61B 1/005; A61B 1/018

(52) U.S. Cl. CPC A61B 1/0057; A61B 1/0052; A61B 1/018

(57) ABSTRACT

The present specification discloses an endoscope with a plurality of steering cables for maneuvering the bending section, some of which are positioned on the internal walls of the bending section using cable guides. Cable stoppers placed in the last vertebra (proximal articulated segment) of the bending section are used to secure the cable close to the distal tip at one end, and to the knobs located in the endoscope handle at the other end. In one embodiment, one of the cable stoppers is located such that it is flanged to the proximal end of the distal tip. This design leaves more free space in the internal diameter of the last vertebra and does not cause obstruction or interference in the proper functioning of other components, such as the front working channel, during the movement of the four steering cables for maneuvering the bending section.

### CROSS-REFERENCE

The present specification relies on, for priority, U.S. Provisional Patent Application No. 61/987,920, entitled “Stopper for the Cables of A Bending Section of An Endoscope”, and filed on May 2, 2014.

The present specification relates to U.S. patent application Ser. No. 14/278,221, entitled “Endoscope Control Unit with Braking System” and filed on May 15, 2014, which relies on, for priority, U.S. Provisional Patent Application Nos. 61/824,634 and 61/837,108, filed on May 17, 2013 and Jun. 19, 2013, respectively.

The above-mentioned applications are herein incorporated by reference in their entirety.

### FIELD

The present specification relates generally to endoscopes, and more specifically, to a stopper for securing a cable used to control the movement of a bending section of an endoscope during an endoscopic procedure.

### BACKGROUND

An endoscope is a medical instrument used for examining and treating internal body parts such as the alimentary canals, airways, the gastrointestinal system, and other organ systems. Conventionally used endoscopes have at least an insertion tube carrying a fiber optic light guide for directing light from an external light source situated at a proximal end of the tube to a distal tip. Also, most endoscopes are provided with one or more channels, through which medical devices, such as forceps, probes, and other tools, may be passed. Further, during an endoscopic procedure, fluids, such as water, saline, drugs, contrast material, dyes, or emulsifiers are often introduced or evacuated via the flexible tube. A plurality of channels, one each for introduction and suctioning of liquids, may be provided within the flexible tube.

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, gastroscopy, laparoscopy, upper gastrointestinal endoscopy among others. Endoscopes may be inserted into the body's natural orifices or through an incision in the skin.

In many endoscopes, the distal end of an insertion tube, or bending section, is capable of being articulated by a steering mechanism that includes a pair of external control wheels/knobs coupled to steering cables mounted inside the insertion tube. Rotation of at least one of the control wheels produces an up or down deflection of the distal tip of the insertion tube while rotation of another control wheel produces a left or right deflection of the insertion tube tip. By operating the two control wheels, the distal end of the insertion tube can be pointed at a desired target within the range of motion of the instrument or maneuvered through a tortuous path of travel.

Further, the control wheels or knobs may be locked through respective braking mechanisms, thereby causing the distal end of the insertion tube to be fixed in a desired position. For example, German patent application DE 20 2011 109 769 U1, filed on Jul. 1, 2011 and assigned to the Applicant of the present specification, discloses an endoscope having an articulation unit. In addition, U.S. patent application Ser. No. 14/278,221, entitled “Endoscope Control Unit with Braking System” and filed on May 15, 2014, describes “a control unit that includes a braking system for fixing the position of an endoscope tip is provided. The control system includes an up-down control knob and a right-left control knob. The brakes are engaged by rotating the control knob itself counter-clockwise from a free-wheeling position. After the brakes have been engaged, a sufficient amount of force applied to the control knobs will move the endoscope tip slightly in the corresponding direction, allowing for fine tuning of tip position after braking.”

The deflection of the articulation unit (also called curvature device), and thus of the distal end of the endoscope is effectuated using a series of cables. In each case, two cables arranged opposite each other on the outer circumference of the articulation unit are connected to form a cable pair. The cable pairs are attached in such a way to the cable drums, that they can be adjusted by rotary knobs, so that the distal end of the articulation unit carries out a movement upwards or downwards (up/down; U-D) or a movement in a direction left or right (right/left; R-L).

Typically, the cables which control the bending section Up/Down and Left/Right movements have stoppers, where a stopper acts as a flange for securing the cable close to the distal tip at one end and for securing the cable close to the knobs located in the handle at the other end. However, when a working channel in the insertion tube is large, one or more of the stoppers may impinge upon the working channel, causing a problem with the use of the channel especially when the bending section is bent during an endoscopic procedure.

Therefore what is needed are newly designed cable stoppers that are relocated so that they do not interfere with the working channels and also secure the cables between the handle of the endoscope and its distal tip in an unobtrusive manner.

### SUMMARY

The present specification discloses an endoscope comprising: a control handle located at a proximal end of the endoscope, said control handle having a plurality of knobs; a substantially cylindrical tip located at a distal end of the endoscope, said substantially cylindrical tip comprising a fluid channeling manifold having an external surface and an internal surface, a front working channel extending through the cylindrical tip, a side-pointing viewing element, and a front-pointing viewing element; an insertion tube, wherein a first end of the insertion tube is attached to the control handle and a second end of the insertion tube is attached to the substantially cylindrical tip, wherein the insertion tube comprises a bending section adapted to be maneuvered by a plurality of steering cables attached to a plurality of positions within the bending section, and wherein at least one of said plurality of steering cables is attached to at least one of said plurality of knobs; and an opening positioned within the external surface of the fluid channeling manifold, said opening extending through to said internal surface, wherein an end of at least one of said plurality of steering cables is positioned through said opening such that it extends past the internal surface and out through said opening to the external surface and wherein a stopper is attached to said end of at least one of said plurality of steering cables extending through said opening to the external surface, said stopper being sized such that it is unable to pass through said opening.

The stopper may be adapted to flange the end of at least one of said plurality of steering cables along a periphery surface of the substantially cylindrical tip of the endoscope.

Each of said plurality of steering cables may be threaded through one or more cable guides distributed along internal walls of the bending section.

Optionally, said plurality of steering cables is equal to four.

The bending section may be movable in an upward, downward, rightward and leftward direction with respect to a reference position of the insertion tube. Each of said four steering cables may be attached to at least one of the plurality of knobs. Additionally, at least one cable stopper may be adapted to secure each of the four steering cables to the plurality of knobs located in the control handle of the endoscope.

Optionally, the bending section comprises a plurality of tubular segments.

An end of one of the four steering cables may be positioned in said opening within the external surface of the fluid channeling manifold wherein ends of the remaining three steering cables are attached to a segment of the bending section closest to the substantially cylindrical tip.

The stopper may have a larger diameter compared to the end of the at least one of the plurality of steering cables.

The present specification also discloses an endoscope comprising: a control handle located at a proximal end of the endoscope; a multiple viewing elements tip located at a distal end of the endoscope, said multiple viewing elements tip comprising a substantially cylindrical external surface and internal surface, a front working channel extending through the cylindrical tip, a side-pointing viewing element, and a front-pointing viewing element; an insertion tube, wherein a first end of the insertion tube is attached to the control handle and a second end of the insertion tube is attached to the multiple viewing elements tip, wherein the insertion tube comprises a bending section adapted to be maneuvered by a plurality of steering cables attached to a plurality of positions within the bending section, and wherein a first end of each of the plurality of steering cables is attached to the control handle; and a groove positioned within the external surface of the multiple viewing elements tip, said groove extending through to said internal surface, wherein a second end of one of said plurality of steering cables is positioned through said groove such that it extends past the internal surface and out through said opening to the external surface and wherein a stopper is attached to said second end, said stopper being positioned within the substantially cylindrical external surface and sized such that it is unable to pass through said opening.

The stopper may be adapted to flange the second end of at least one of said plurality of steering cables along a distal front face of the substantially cylindrical tip of the endoscope.

Each of said plurality of steering cables may be threaded through one or more cable guides distributed along internal walls of the bending section.

Optionally, said plurality of steering cables is equal to four.

The bending section may be movable in an upward, downward, rightward and leftward direction with respect to a reference position of the insertion tube.

Optionally, the control handle comprises a plurality of knobs wherein each of said four steering cables is attached to at least one of said plurality of knobs. Additionally, at least one cable stopper may be adapted to secure each of the four steering cables to the plurality of knobs located in the control handle of the endoscope.

An end of one of the four steering cables may be positioned in said groove within the external surface of the multiple viewing elements tip wherein ends of the remaining three steering cables are attached to a segment of the bending section closest to the multiple viewing elements tip.

The stopper may have a larger diameter compared to the second end of the at least one of the plurality of steering cables.

The present specification also discloses an endoscope comprising: a control handle located at a proximal end of the endoscope; a substantially cylindrical multiple viewing elements tip located at a distal end of the endoscope, said substantially cylindrical multiple viewing elements tip comprising a front working channel extending through the cylindrical tip, a side-pointing viewing element, a front-pointing viewing element positioned within a distal face of the multiple viewing elements tip, and a proximal end of the substantially cylindrical multiple viewing elements tip; an insertion tube, wherein a first end of the insertion tube is attached to the control handle and a second end of the insertion tube is attached to the proximal end of the multiple viewing elements tip, wherein the insertion tube comprises a bending section adapted to be maneuvered by a plurality of steering cables attached to a plurality of positions within the bending section, and wherein a first end of each of the plurality of steering cables is attached to the control handle; and a space positioned within an external surface of the proximal end of the multiple viewing elements tip, said space extending through to an internal surface of the multiple viewing elements tip, wherein a second end of one of said plurality of steering cables is positioned through said space such that it extends past the internal surface and out through said space to the external surface and wherein a stopper is attached to said second end, said stopper being positioned within the proximal end of the multiple viewing elements tip and sized such that it is unable to pass through said opening.

The aforementioned and other embodiments of the present shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates a multiple viewing elements endoscopy system, in accordance with an embodiment of the present specification;

FIG. 2 illustrates a perspective view of a multiple viewing elements endoscope, in accordance with an embodiment of the present specification;

FIG. 3A illustrates a perspective view of a bending section of a multiple viewing elements endoscope, in accordance with an embodiment of the present specification;

FIG. 3B illustrates another view of the bending section shown in FIG. 3A;

FIG. 3C illustrates a perspective view of a tubular segment of the bending section shown in FIG. 3A;

FIG. 4 illustrates a cross-sectional view of a bending section of a multiple viewing elements endoscope, in accordance with an embodiment of the present specification;

FIG. 5 illustrates a perspective view of the rear side of the tip section with a groove for cable stopper, in accordance with one embodiment of the present specification;

FIG. 6 shows a detail of the distal tip, with the cable stopper placed in the designated groove, according to one embodiment of the present specification;

FIG. 7 illustrates a perspective view of a segment of a bending section, showing the conventional location for cable stoppers; and

FIG. 8 provides a perspective view of the distal tip showing the cable stopper in its designated groove, according to one embodiment of the present specification.

### DETAILED DESCRIPTION

In some embodiments, the present specification discloses an endoscope including a handle from which a proximal end of an elongated insertion tube emerges, whereby a tip section is positioned at the distal end of the insertion tube, and wherein the tip section comprises multiple viewing elements and illuminators. The insertion tube and thus tip section is turnable by way of a bending portion of the insertion tube that is connected at a proximal end of the tip section. Thus, a proximal end of the bending portion is located at a distal end of the insertion tube and a distal end of the bending portion is connected to a proximal end of the tip section. In an embodiment, the endoscope comprises a plurality of steering cable guides or eyes, positioned on the internal walls of the bending section. Steering cables are threaded through these guides or eyes, to enable the maneuvering of the bending portion of the insertion tube. In an embodiment, the handle is used for maneuvering the insertion tube within a body cavity by means of one or more operating knobs which control the movement of the bending section. In an embodiment, the operating knobs enable a directional readjustment of right and left (or up and down) movement of the endoscope tip by controlling the movement of the bending section.

In some embodiments, stopper(s) are provided on the steering cables that control the movement of the bending section, where the stopper(s) act as flanges for securing the cable close to the tip section at the distal end of the endoscope (also interchangeably referred to as the “distal tip”) and for securing the cable close to the knobs located in the handle at the proximal end of the endoscope. In some embodiments, the location and position of the stoppers are designed such that they do not interfere with the working channels and secure the cables between the handle of the endoscope and its distal tip in an unobtrusive manner.

In an embodiment of the present specification, an endoscope is provided with four steering cables for maneuvering the bending section of the insertion tube. The cables are positioned on the internal walls of the bending section using cable guides. Cable stoppers are placed in the last vertebra of the bending section, located at a distal end of the bending section, and are used to secure the cables close to the tip section at the distal end of the insertion tube and close to the knobs located in the endoscope handle at the proximal end of the insertion tube.

In an embodiment, at least one of the cable stoppers is optionally positioned such that it is flanged to the proximal end of the tip section, instead of the last vertebra at the distal end of the bending section. This design leaves more free space within the internal diameter of the bending section/last vertebra and does not cause obstruction or interference in the proper functioning of other components, such as the front working channel, during the movement of the four steering cables that maneuver the bending section.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

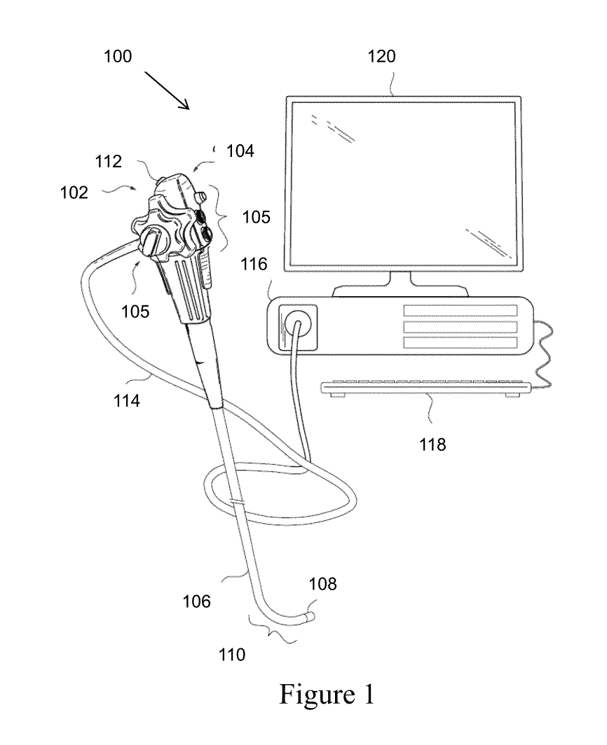

Reference is now made to FIG. 1, which shows a multiple viewing elements endoscopy system 100. System 100 comprises a multiple viewing elements endoscope 102. Multiple viewing elements endoscope 102 comprises a handle 104, from which an elongated insertion tube 106 extends. Elongated insertion tube 106 terminates with a tip section 108 which is turnable by way of a bending section 110. Tip 108 comprises one or more viewing elements such as cameras, sensors and one or more illuminators. Handle 104 may be used for maneuvering insertion tube 106 within a body cavity. Handle 104 comprises one or more knobs and/or switches 105 which control bending section 110 as well as functions such as fluid injection and suction. Handle 104 may further include a working channel opening 112 through which surgical tools may be inserted.

A utility cable/umbilical tube 114 may connect between handle 104 and a main control unit 116. Umbilical tube 114 comprises therein one or more fluid channels and one or more electrical channels. The electrical channel(s) may include at least one data cable for receiving video signals from the one or more viewing elements, as well as at least one power cable for providing electrical power to the viewing elements and to the illuminators.

One or more input devices, such as a keyboard 118, may be connected to main control unit 116 for the purpose of human interaction with the main control unit 116. A display 120 may be connected to main control unit 116, and configured to display images and/or video streams received from the viewing elements of multiple viewing elements endoscope 102.

In an embodiment, the tip 108 of the insertion tube 106 is capable of being articulated by a means of an articulation unit that controls the bending movement of the bending section 110. United States Patent Application Number 2014/0343489, entitled “Endoscope Control Unit with Braking System” and filed on May 15, 2014, assigned to the Applicant of the present specification, herein incorporated by reference in its entirety, provides an example of an articulation unit.

In an embodiment, a plurality of steering cable guides (also referred to as eyes) is positioned on the internal walls of bending section 110. Steering cables are threaded through these guides to enable the maneuvering of bending section 110. In an embodiment, the articulation unit comprises a pair of external control wheels/knobs 105 coupled to the steering cables. In an embodiment, two steering cables arranged opposite each other on the outer circumference of the articulation unit are connected to form a cable pair. At least one of the knobs 105 is connected to a cable drum in a manner such that rotating/turning the knob results in a rotation of the cable drum pulling the cable pair, which, in turn, pulls the cable pair attached to the bending section 110 and consequently maneuvers the bending section 110 accordingly.

Rotation of at least one knob 105 produces an up or down deflection of the tip 108 of the insertion tube 106 while rotation of at least one knob 105 produces a left or right deflection of the insertion tube tip 108. By operating the knobs 105, the distal end of the insertion tube 106 comprising the bending section 110 and the tip 108 can be pointed at a desired target within the range of the endoscope or maneuvered through a tortuous path of travel. Further, the knobs used for bending the bending section 110 may be locked through respective braking mechanisms, as described in the applications referenced above, thereby causing the tip 108 to be fixed in a desired position.

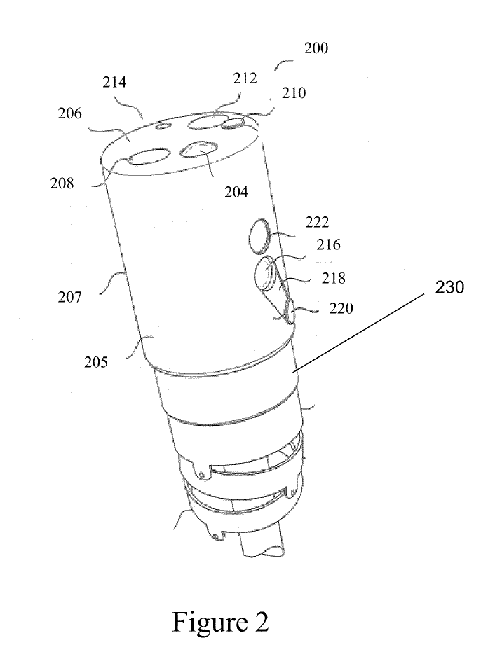

Reference is now made to FIG. 2, which shows a perspective view of the distal face of a tip section and a portion of a bending section of a multiple viewing elements endoscope 200, in accordance with an embodiment of the present specification. Tip section 207 of the endoscope 200 includes a front-pointing viewing element 204 for capturing images through an opening in a distal end face/surface 206 of the tip section. Tip section 207 has a proximal end 230 connected to a distal end of the bending section of the insertion tube. A discrete front illuminator 208, which is in an embodiment a light-emitting diode (LED), associated with front-pointing viewing element 204 is used for illuminating its field of view through another opening in the distal end face/surface 206.

A front fluid injector 210 is used for cleaning at least one of front-pointing viewing element 204 and discrete front illuminator 208. Distal end surface 206 further includes an opening defining a working channel 212, which may be a hollow tube configured for insertion of a surgical tool to operate on various tissues. A pathway fluid injector 214, defined by another opening in distal end surface 206, is used for inflating and/or cleaning the body cavity into which endoscope 200 is inserted. Tip section 207 further comprises a side-pointing viewing element 216 used for capturing images through an opening in a cylindrical surface 205 of the tip section. A discrete side illuminator 222, which is optionally similar to discrete front illuminator 208, in one embodiment, is associated with front-pointing viewing element 204 and used for illuminating its field of view through another opening in cylindrical surface 205.

A side fluid injector 220 is used for cleaning at least one of side-pointing viewing element 216 and discrete side illuminator 222. In order to prevent tissue damage when cylindrical surface 205 of tip section 207 contacts a side wall of the body cavity, side fluid injector 220 and side-pointing viewing element 216, in one embodiment are located in a depression 218 in the cylindrical surface. In an alternative configuration, one or more discrete side illuminators may also be included in the depression, so that fluid injected from the side fluid injector reaches them. In yet another configuration, a side-pointing viewing element, one or more side illuminators and a side fluid injector may be positioned on essentially the same level as the cylindrical surface of the tip section.

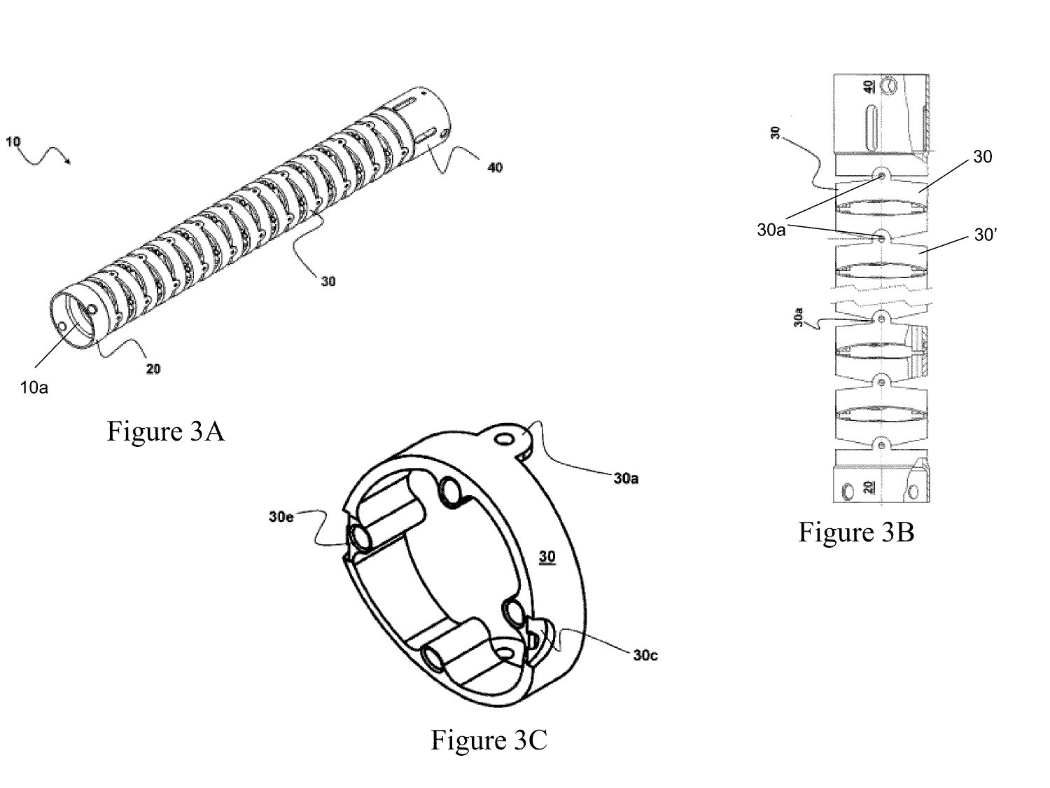

FIG. 3A illustrates a perspective view of a bending section of a multiple viewing elements endoscope, in accordance with an embodiment of the present specification. The bending section 10 comprises a plurality of mutually articulated tubular segments 20, 30, 40. In an embodiment, proximal articulated segment 20 and distal articulated segment 40 are constructed differently than segment 30. Optionally, the bending section 10 comprises at least one proximal segment 20, at least one segment 30, and at least one distal segment 40. Optionally, the bending section 10 comprises one proximal segment 20, one distal segment 40 and a plurality of segments 30. A lumen 10a runs through the bending section 10.

FIG. 3B illustrates another view of the bending section shown in FIG. 3A. As is shown, segment 30 comprises at least one tubular part. The at least one tubular part comprises two oppositely oriented axially extending tabs 30a positioned along a first periphery and one or more recesses positioned along a second opposing periphery. The segments 30 are coupled together by tabs 30a of a first tubular part which fits into a recess of an adjacent second tubular part. For example, as shown in FIG. 3B, tab 30a of first part 30 fits into a recess of second part 30′, and so forth, thereby coupling segment parts 30.

FIG. 3C illustrates a perspective view of the segment 30 of the bending section, shown in FIG. 3B. In an embodiment, segment 30 comprises one or more axially extending tabs 30a positioned along a distal periphery. In an embodiment, segment 30 comprises one or more recesses 30c positioned along a proximal periphery for coupling with extending tabs 30a of segment 30. Thus, one or more axially extending tabs 30a are provided on a first segment 30 for coupling with a recess 30c of a second, adjacent segment 30. Cable guides (also referred to as ‘eyes’ or ‘eyelets’) 30e are positioned along internal walls of segment 30, extending into lumen 10a (shown in FIG. 3A). In one embodiment, cable guides are made up of metal and are welded into the internal walls of the circular section of segment 30. In various embodiments one or more steering cables may be threaded through these cable guides to enable the maneuvering of bending section 10.

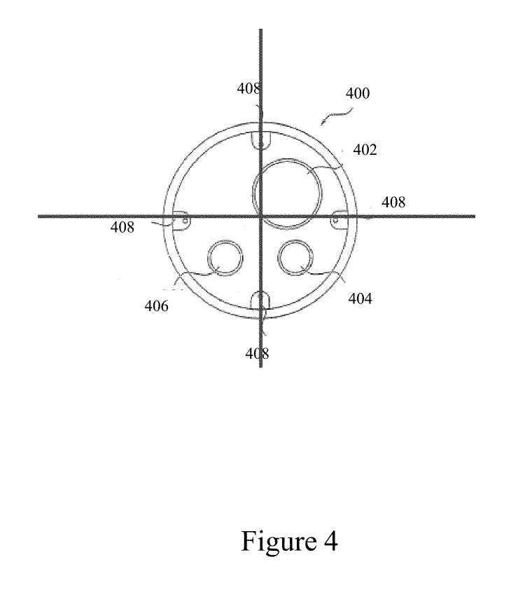

Reference is now made to FIG. 4, which shows a cross-sectional view of a bending section 400 of a multiple viewing elements endoscope, such as multiple viewing elements endoscope 100 of FIG. 1. A plurality of eyelets 408 are positioned on the internal walls of bending section 400. In one embodiment, four eyelets are employed along the length of bending section 400. It may be noted that the figure shows one embodiment, wherein four eyelets are placed at angles of 90 degrees relative to each other. It may be appreciated, however that the eyelets may be located at angles smaller or larger relative to each other, than that shown in the figure. It should also be understood that while four eyelets are described with respect to the present specification, any number of eyelets may be employed. Steering cables are threaded through these eyelets 408 to enable the maneuvering of bending section 400.

Bending section 400, in an embodiment, comprises a working channel 402, through which surgical tools are inserted, a fluid channel 406, through which fluids and/or liquids are infused, and an electrical channel 404 with a plurality of electrical cables threaded through it, for transmitting video signals from the viewing elements and for supplying power to the viewing elements and the discrete illuminators. In one embodiment, bending section 400 comprises more than one working channel, more than one fluid channel and more than one electrical channel.

Typically the four cables which control the bending section Up/Down and Left/Right movements have stoppers, where each stopper acts as a flange to secure the cable close to the tip at the distal end of the insertion tube and to the knobs located in the handle at the proximal end of the insertion tube. Referring back to FIGS. 3A and 3B, the stoppers are conventionally located in the proximal articulated segment 20.

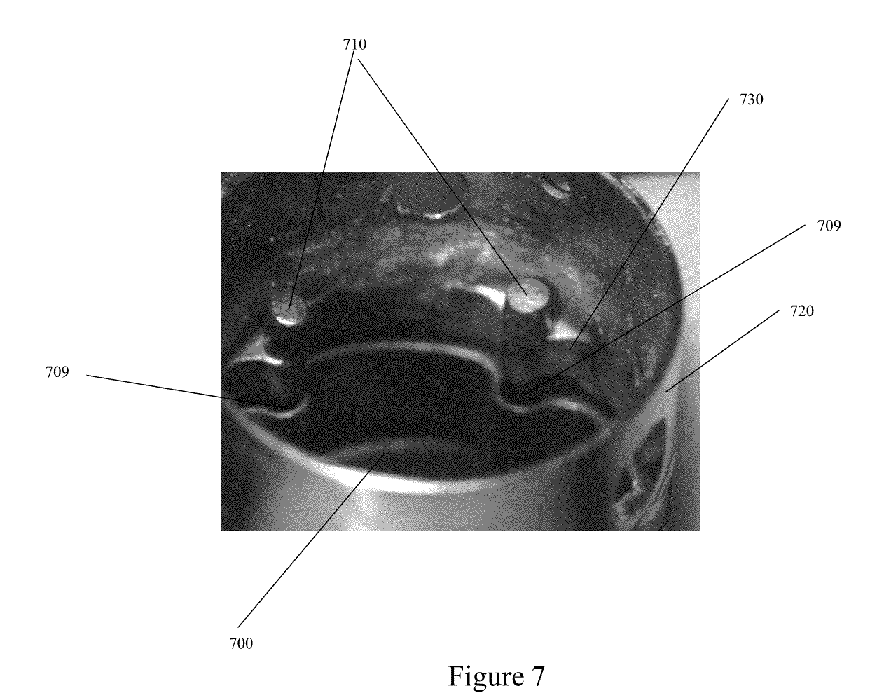

FIG. 7 provides a perspective view, illustrating the convention location of the cable stoppers. Referring to FIG. 7, a proximal articulated segment 720 of the bending section of the insertion tube 700 is shown. The bending section comprises steering cables 709, with cable stoppers 710 coupled to corresponding steering cables. Cable guides or ‘eyelets’ 730 are positioned along internal walls of segment 720. As seen in the figure, steering cables, which enable the maneuvering of bending section, are threaded through the cable guides. A cable stopper 710 is made up of metal such as tin, and is generally welded into the steering cable 709. The stopper acts to anchor the cable it to its place and limits the cable movement.

A problem arises however, when any of the working channels, including fluid channel or electrical channel, as shown in FIG. 4, have a large diameter. In this case, a cable stopper may impinge upon a working channel, especially when the bending section is bent during an endoscopic procedure. For example, if the front working channel has a diameter of 2.9 mm or more, one of the stoppers may press on the working channel and, as a result, interfere with its functionality.

The above problem is addressed, in one embodiment, by moving at least one of the stoppers from the proximal articulated segment of the bending section of the insertion tube and relocating it in the distal tip. This prevents any obstruction or impingement on the working channel.

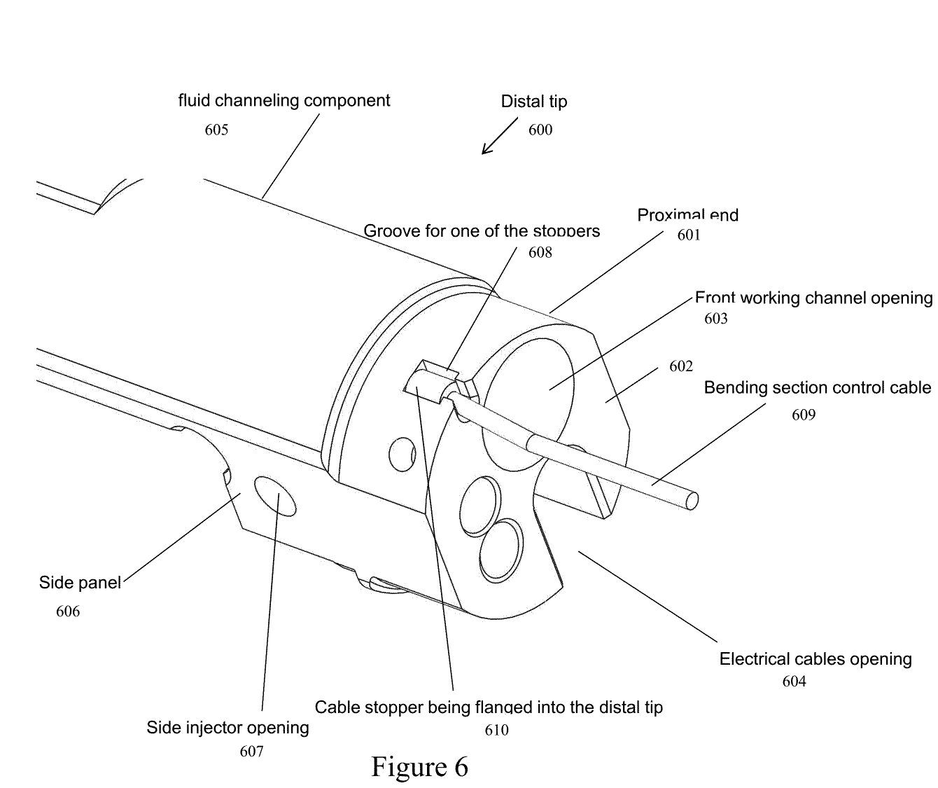

It may be appreciated that the distal ends of four cables which control the movement of the bending section are typically flanged to the last vertebra (proximal articulated segment) of the bending section (shown as component 20 in FIG. 3A), with the help of corresponding cable stoppers. It may further be appreciated that the diameter of the last vertebra of the bending section is small, and various channels and components, including working channel, jet, injector, electronic cables, etc. enter the distal tip from the last vertebra. In such a case, having four cable stoppers in the last vertebra leads to crowding and may cause obstruction or interference in the proper function of other components. The present specification provides a solution to these architectural constraints by re-locating one of the stoppers such that it is flanged to the distal tip, instead of the last vertebra of the bending section. This design leaves more free space in the internal diameter of the last vertebra (component 20 of FIG. 3A), as the stopper has a larger diameter compared to the steering cable (shown in FIG. 6). This relocation of the stopper also ensures that there is no obstruction or impingement on the working channel or other components during the movement of the four steering cables for maneuvering the bending section.

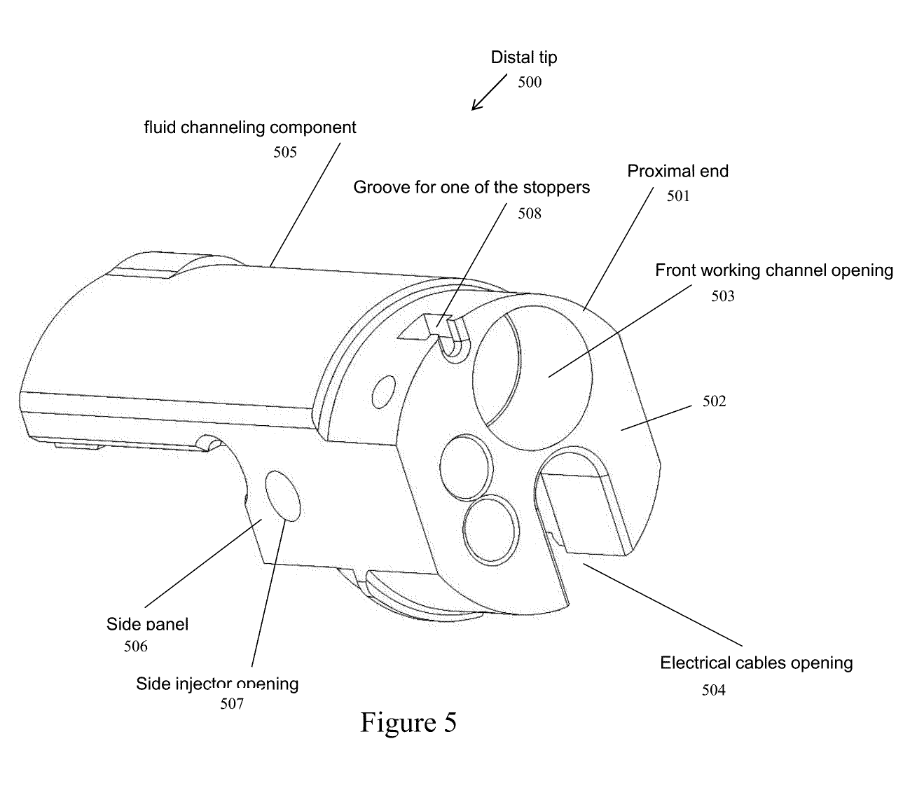

FIG. 5 illustrates a novel design for a tip to incorporate a novel positioning of a stopper in a perspective view of the rear side of the tip section, in accordance with one embodiment of the present specification. Referring to FIG. 5, the distal tip 500 comprises a rear panel 502. The rear panel 502 comprises a front working channel opening 503 and another opening 504 for electrical cables. The rear panel 502 is coupled to a fluid channeling component/manifold 505 and a side panel 506. Side panel 506 further comprises a side injector opening 507, which is used for cleaning the side-pointing viewing element(s) and discrete side illuminator(s). In one embodiment, a groove 508 for receiving one of the steering cable stoppers is positioned in the proximal end 501 of the fluid channeling component 505.

FIG. 6 shows a detail of the distal tip 600, with a cable stopper 610 placed in the designated groove 608. As seen in the figure, the rear panel 602 comprises a front working channel opening 603 and another opening 604 for electrical cables. The rear panel 502 is coupled to a fluid channeling component 605 and a side panel 606. Side panel 606 further comprises a side injector opening 607. The proximal end 601 of the fluid channeling component 605 comprises a groove 608 with the cable stopper 610 placed inside. The stopper 610 is connected to a steering cable 609, which is used to control the movement of the bending section of the insertion tube. The purpose of the groove 608 is to anchor the cable's stopper 610. In one embodiment, the opening diameter of the groove is just large enough to accommodate the cable but not larger than the stopper diameter, which, because the stopper is welded, brazed, fused, melted onto, or molded to the end of the cable, ensures that the cable does not get pulled out. As explained earlier, steering cable runs from the knobs of the endoscope handle into the insertion tube, and is flanged to proximal end of the distal tip using the stopper 610. In one embodiment, the cable stopper 610 is made up of metal, such as tin, and is welded or fused by melting into the cable. One of ordinary skill in the art would appreciate that any known method for placing and coupling the stopper to the cable may be used.

In one embodiment, the endoscope comprises four steering cables with one of the four steering cables secured to the outside surface, or periphery, of the distal tip and the remaining three steering cables attached to a portion of the bending section closest to the distal tip. In one embodiment, the endoscope comprises four steering cables with two of the four steering cables secured to the outside surface, or periphery, of the distal tip and the remaining two steering cables attached to a portion of the bending section closest to the distal tip. In one embodiment, the endoscope comprises four steering cables with three of the four steering cables secured to the outside surface, or periphery, of the distal tip and the remaining one steering cable attached to a portion of the bending section closest to the distal tip. In one embodiment, the endoscope comprises four steering cables with all four steering cables secured to the outside surface, or periphery, of the distal tip and no steering cables attached to a portion of the bending section closest to the distal tip. In one embodiment, the endoscope comprises more than four steering cables with one of those steering cables secured to the outside surface, or periphery, of the distal tip and the remaining steering cables attached to a portion of the bending section closest to the distal tip. In one embodiment, the endoscope comprises less than four steering cables with one of those steering cables secured to the outside surface, or periphery, of the distal tip and the remaining steering cables attached to a portion of the bending section closest to the distal tip.

As known in the art, when the cable is in its traditional location in the last vertebra of the bending section, the stopper which is typically made of metal such as tin, anchors the cable to its place and limits the cable movement. In the new design of the present embodiment, as shown in FIG. 6, one of the stoppers is placed on the edge of the cable, and is located in groove 608 for anchoring the cable 609. In one embodiment, a tip cover (not shown) is provided above the distal tip to ensure sealing of the stopper in its place.

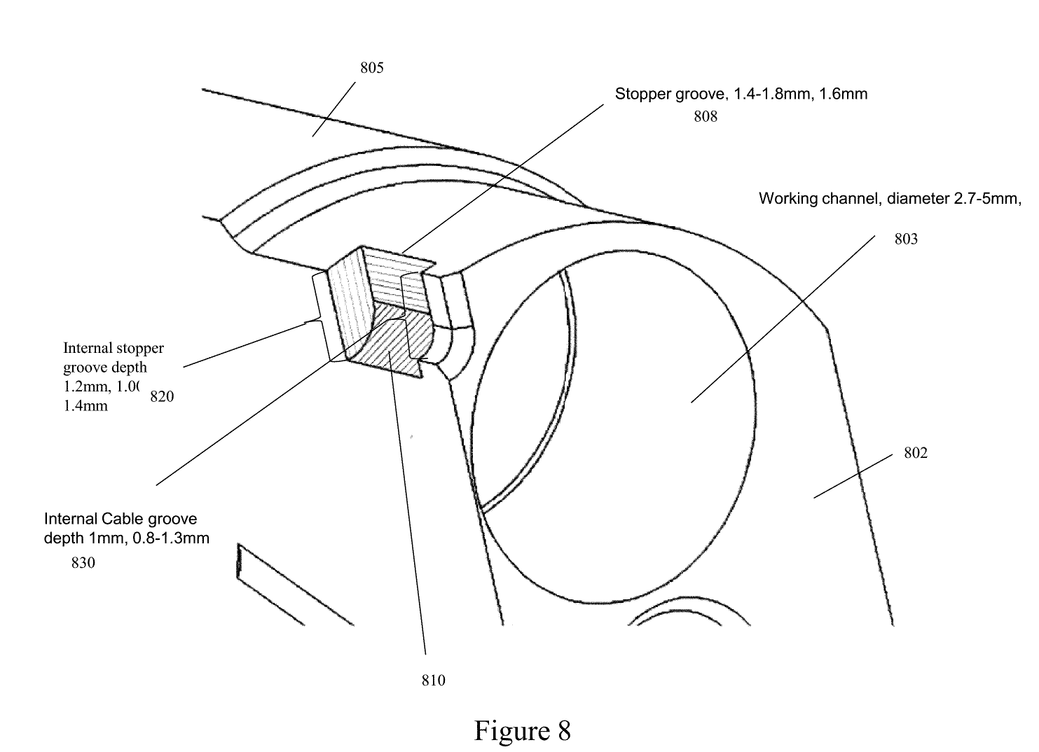

FIG. 8 provides another perspective view of the distal tip showing the cable stopper in its designated groove. Referring to FIG. 8, the rear panel 802 of a distal tip is shown, which comprises a front working channel opening 803. In one embodiment, the working channel diameter ranges from 2.5 to 5 mm. The rear panel 802 is coupled to a fluid channeling component 805, which further comprises a groove 808 with the cable stopper 810 placed inside. In one embodiment, as shown in FIG. 8, the groove and stopper are located on a surface of the tip which is on the periphery of a side of the fluid channeling component of the tip section. In another embodiment (not shown), the groove is located on the front, distal surface of the tip, or on the front of rear panel 802. In one embodiment, the groove extends from the outer surface of the tip section and extends into its distal face. That is, the groove is partially positioned on the external periphery. It may be appreciated that the groove may be located anywhere on proximal end of the tip section, which is connected to a distal end of the bending section of the insertion tube (as shown in FIG. 2), as long as it efficiently anchors the steering cable.

In one embodiment, the width of the stopper groove 808 ranges from 1.4 mm to 1.8 mm, and the internal depth 820 of the groove ranges from 1.00 mm to 1.4 mm. In one embodiment, the depth 830 of the internal hole within the stopper, which allows the steering cable to pass through ranges from 0.8 mm to 1.3 mm. In one embodiment, the opening of the stopper groove is large enough to accommodate the cable but not larger than the stopper diameter, which ensures that the cable does not get pulled out. Operationally, the system is manufactured by passing a cable through each of the eyelets within the bending section and through the groove positioned on the periphery of the distal tip. Once passed through the groove, the stopper is attached, via welding, brazing, fusing, melting, or molding, to the cable end to secure the cable into the groove. Alternatively, the system is manufactured by first making a cable with a stopper attached, via welding, brazing, fusing, melting, or molding, to one end of the cable end. The free end of the cable is then threaded through the groove positioned on the periphery of the distal tip and then through each of the eyelets within the bending section.

The above examples are merely illustrative of the many applications of the system of present specification. Although only a few embodiments of the present specification have been described herein, it should be understood that the present specification might be embodied in many other specific forms without departing from the spirit or scope of the invention. Therefore, the present examples and embodiments are to be considered as illustrative and not restrictive.

We claim:

1. An endoscope comprising:
a control handle located at a proximal end of the endoscope, said control handle having a plurality of knobs;
a substantially cylindrical tip located at a distal end of the endoscope, said substantially cylindrical tip comprising a fluid channeling manifold having an external surface and an internal surface, a front working channel extending through the cylindrical tip, a side-pointing viewing element, and a front-pointing viewing element;
an insertion tube, wherein a first end of the insertion tube is attached to the control handle and a second end of the insertion tube is attached to the substantially cylindrical tip, wherein the insertion tube comprises a bending section adapted to be maneuvered by a plurality of steering cables attached to a plurality of positions within the bending section, and wherein at least one of said plurality of steering cables is attached to at least one of said plurality of knobs; and
an opening positioned within the external surface of the fluid channeling manifold, said opening extending through to said internal surface, wherein an end of at least one of said plurality of steering cables is positioned through said opening such that it extends past the internal surface and out through said opening to the external surface and wherein a stopper is attached to said end of at least one of said plurality of steering cables extending through said opening to the external surface, said stopper being sized such that it is unable to pass through said opening.

2. The endoscope of claim 1, wherein said stopper is adapted to flange the end of at least one of said plurality of steering cables along a periphery surface of the substantially cylindrical tip of the endoscope.

3. The endoscope of claim 1, wherein each of said plurality of steering cables is threaded through one or more cable guides distributed along internal walls of the bending section.

4. The endoscope of claim 1, wherein said plurality of steering cables is equal to four.

5. The endoscope of claim 1, wherein the bending section is movable in an upward, downward, rightward and leftward direction with respect to a reference position of the insertion tube.

6. The endoscope of claim 4, wherein each of said four steering cables is attached to at least one of the plurality of knobs.

7. The endoscope of claim 6, wherein at least one cable stopper is adapted to secure each of the four steering cables to the plurality of knobs located in the control handle of the endoscope.

8. The endoscope of claim 1, wherein the bending section comprises a plurality of tubular segments.

9. The endoscope of claim 6, wherein an end of one of the four steering cables is positioned in said opening within the external surface of the fluid channeling manifold and wherein ends of the remaining three steering cables are attached to a segment of the bending section closest to the substantially cylindrical tip.

10. The endoscope of claim 1, wherein the stopper has a larger diameter compared to the end of the at least one of the plurality of steering cables.

11. An endoscope comprising:
a control handle located at a proximal end of the endoscope;
a multiple viewing elements tip located at a distal end of the endoscope, said multiple viewing elements tip comprising a substantially cylindrical external surface and internal surface, a front working channel extending through the cylindrical tip, a side-pointing viewing element, and a front-pointing viewing element;
an insertion tube, wherein a first end of the insertion tube is attached to the control handle and a second end of the insertion tube is attached to the multiple viewing elements tip, wherein the insertion tube comprises a bending section adapted to be maneuvered by a plurality of steering cables attached to a plurality of positions within the bending section, and wherein a first end of each of the plurality of steering cables is attached to the control handle; and
a groove positioned within the external surface of the multiple viewing elements tip, said groove extending through to said internal surface, wherein a second end of one of said plurality of steering cables is positioned through said groove such that it extends past the internal surface and out through said opening to the external surface and wherein a stopper is attached to said second end, said stopper being positioned within the substantially cylindrical external surface and sized such that it is unable to pass through said opening.

12. The endoscope of claim 11, wherein said stopper is adapted to flange the second end of at least one of said plurality of steering cables along a distal front face of the substantially cylindrical tip of the endoscope.

13. The endoscope of claim 11, wherein each of said plurality of steering cables is threaded through one or more cable guides distributed along internal walls of the bending section.

14. The endoscope of claim 11, wherein said plurality of steering cables is equal to four.

15. The endoscope of claim 11, wherein the bending section is movable in an upward, downward, rightward and leftward direction with respect to a reference position of the insertion tube.

16. The endoscope of claim 14, wherein the control handle comprises a plurality of knobs and wherein each of said four steering cables is attached to at least one of said plurality of knobs.

17. The endoscope of claim 16, wherein at least one cable stopper is adapted to secure each of the four steering cables to the plurality of knobs located in the control handle of the endoscope.

18. The endoscope of claim 14, wherein an end of one of the four steering cables is positioned in said groove within the external surface of the multiple viewing elements tip and wherein ends of the remaining three steering cables are attached to a segment of the bending section closest to the multiple viewing elements tip.

19. The endoscope of claim 11, wherein the stopper has a larger diameter compared to the second end of the at least one of the plurality of steering cables.

20. An endoscope comprising:
a control handle located at a proximal end of the endoscope;
a substantially cylindrical multiple viewing elements tip located at a distal end of the endoscope, said substantially cylindrical multiple viewing elements tip comprising a front working channel extending through the cylindrical tip, a side-pointing viewing element, a front-pointing viewing element positioned within a distal face of the multiple viewing elements tip, and a proximal end of the substantially cylindrical multiple viewing elements tip;
an insertion tube, wherein a first end of the insertion tube is attached to the control handle and a second end of the insertion tube is attached to the proximal end of the multiple viewing elements tip, wherein the insertion tube comprises a bending section adapted to be maneuvered by a plurality of steering cables attached to a plurality of positions within the bending section, and wherein a first end of each of the plurality of steering cables is attached to the control handle; and
a space positioned within an external surface of the proximal end of the multiple viewing elements tip, said space extending through to an internal surface of the multiple viewing elements tip, wherein a second end of one of said plurality of steering cables is positioned through said space such that it extends past the internal surface and out through said space to the external surface and wherein a stopper is attached to said second end, said stopper being positioned within the proximal end of the multiple viewing elements tip and sized such that it is unable to pass through said opening.
